(19) United States
(12) Patent Application Publication
(10) Pub. No.: US 2001/0047162 A1
(43) Pub. Date: Nov. 29, 2001

(54) Injection kit and injection device

(76) Inventor: Yasumi Yugari, Kamakura-shi (JP)

Correspondence Address: OBLON SPIVAK MCCLELLAND MAIER & NEUSTADT PC, FOURTH FLOOR, 1755 JEFFERSON DAVIS HIGHWAY, ARLINGTON, VA, 22202, US

(21) Appl. No.: 09/783,998

(22) Filed: Feb. 16, 2001

(30) Foreign Application Priority Data
Feb. 17, 2000 (JP) 2000-039368

(51) Int. Cl. B65D 30/22; A61B 19/00

(52) U.S. Cl. 604/410; 604/411; 206/219

(57) ABSTRACT

In this invention, injection kit is to provide. The kit is made of multiple layered flexible plastic bag formed cylindrically (soft bag), and is separated into 2˜4 compartments by one or plural welded partition easy-to-peel seal. The first compartment has a welded nozzle-like outlet of the injection solution and contains a solid medicine. The rearmost compartment (the last compartment of this kit) contains a solvent to dissolve the solid medicine. The remaining one or two mid-compartment contains a solid medicine and/or its solvent. The welded nozzle-like outlet of the first compartment is welded with a metallic injection needle, which is covered by a plastic protective cap before it is used.

Above mentioned soft bag provides the plural small holes on the outside and opposite to the outlet that get into the plural cylindrical projections placed on the rear part of a saucer of the injection device in order to set the soft bag to the injection device.

In this invention, manufacturing process of the injection kit is also described.

Through this invention, preparation of injection solution is put into practice by very simple mechanism and procedure, minimizing the chance of infection even under the unsanitary environment, and the solution thus prepared can be injected to patient directly from the bag through injection needle provided to the bag. This invention also minimizes the number of materials required for the preparation and injection of drugs, and thus, the custody of pharmacy is easier.

This invention supplies an injection kit made only of plastic and metal (injections needle). This procedure limits the medical waste into 2 kinds, and also supplies injection device, which is very advantageous for the practice of injection with this injection kit.

### BACKGROUND OF THE INVENTION

1. Technological Field to which this Invention Belongs

Firstly, this invention concerns to an injection kit.

For further information, the plastic soft bag is separated into two compartments by a welded partition easy-to-peel seal. A small quantity of a solid medicine, for example about 0.5˜3 g, is filled in one compartment and a solvent for the solid medicine, for example about 1˜20 ml, is filled in another. At the one side of the solid medicine compartment, a nozzle-like outlet of the injection solution is placed and a metallic needle is welded on it. Thus this invention concerns to an injection kit which enables to inject to a patient directly upon the preparation of the solution with the kit. The soft bag can be consisted from 3 or 4 compartments by welded partition easy-to-peel seal, if necessary (as mentioned later).

This invention also concerns to the preservation of its airtightness by welding or attaching at the partition point of the bag easy-to-peel seal to separate solid medicine compartment and the solvent compartment. For the purpose of keeping its quality from deterioration upon moisture absorption, the medicine compartment and the metallic needle with a cap are totally covered by a sheath-shaped bag, to be kept dry.

The sheath-shaped bag, described above, with the moisture-proofing purpose, is made by a multi-layer film consisting of a transparent gas-barrier plastic layer and a soft, low melting-point plastic layer. A certain amount of desiccant is either applied or attached between the multi-layer films described above in one side, so as not to prevent the observation of the inside of the sheath-shaped bag. Or a desiccant powder is filled inside, or a piece of a paper containing desiccant lies between the multi-layer films. A metallic foil can replace a part gas-barrier plastic layer, as long as the inner condition of a sheath-shaped bag is observed. Again, the soft, low melting point plastic layer welded as an inner layer. At the use of a desiccant, it can be placed in the space made by the sheath-shaped moisture-proofing bag, instead of placed between the two layers of the multi-layer films.

The moisture-proofing bag manufactured under the process above, which is welded or attached to an injection kit, can be removed when an injection kit is set to an injection device that will be described later.

Secondly, this invention concerns to an injection device.

For further information, this invention also concerns to an injection device which enables to inject directly to a patient, by setting an injection kit to an injection device consisted of a lid and a saucer. Then, by pressing the soft bag with block and rod and moving it forward gently, a welded partition of easy-to-peel seal is broken so that the injection solution can flow into a solid medicine compartment. Thus, a solid medicine is dissolved in the closed soft bag. Upon the preparation of the injection solution, the cap covering the metallic needle is removed. By pushing the press block forward smoothly, the air remaining inside of the soft bag is pushed out through the needle. For the injection, pushing the rod further; the soft bag is pressed to push out the injection solution.

In other words, this invention concerns to an injection kit and an injection device, which enable to prepare an injection solution speedily, easily, safely and aseptically, by dissolving a solid medicine for intravenous, intra-muscular, hypodermic and intra-dermal injections. The operator should prepare it without the error to mistake the combination of a solid medicine and its solvent and/or their respective quantities. It also enables to prepare it without the fear of the operator suffering cut wound by a piece of broken glass of a glass vial or ampoule. There is no necessity to choose the place for preparation including even in the fields. The injection solution can be prepared exactly the same and quality unrelated to the skillfulness of the operator. Thus, the injecting dosage can be ready to inject in a closed bag furnishing injection needle without the necessity of transferring the solution into another glass or a plastic injector.

2. Prior Art

The hitherto known container for the injection solution is a glass or a hard plastic ampoule and a vial. Especially, when a solid medicine is unstable in the solute condition, when it deteriorate upon its moisture absorption, and when it has a bad solubility, it is usually prepared in a glass vial by freeze-drying, while its solvent is sealed in another glass ampoule. Therefore to perform the injection, one should cut a glass ampoule, set a disposable metallic injection needle to a disposable syringe to suck a certain amount of the solvent from the ampoule into the syringe. Then, one should strip off a protective seal on a rubber stopper of the glass vial wipe it with some cotton wetted by alcohol, inject the solvent into the vial by piercing the rubber stopper to dissolve a solid medicine. Then, suck again the prepared solution into the syringe. And thus, the whole arrangement of the injection is completed.

A flexible container made of a soft plastic, namely so-called a soft bag, is known as a container for a medicine of the injection. Compared with a glass container, the advantages of using a soft bag as a container can be described as follows; a soft bag suffers less damage, it is light in weight, and it can be easily disposed after the usage and so on. Therefore, it is suitable for the storage of large volume of a medicine, and it is widely used as a container for infusion solutions.

Lately, the soft bag which has some compartments separated by welded partition easy-to-peel seals, has been worked out. Thus, two or more kinds of liquid medicine that might have possibilities of changing their own qualities when mixed can be filled in different compartment. At the time of usage, pushing the solvent compartment with palm breaks partition seal for mixing. In this way, it has been very conveniently used, especially useful when the dosage of the mixed solution, consisting of an electrolyte and glucose, and an amino acids solution.

When a soft bag is used as a container of liquid medicine, a port that works as an outlet of the liquid is prepared in the soft bag. Rubber stopper seals it, and an injection needle connected to tubing for infusion pierces the stopper at the time of usage. It is general that the injection needle on the other end of the infusion tubing is pierced into a patient's vein for the intravenous drip.

The soft bag consisting of plural separated compartments, which are separated by welded partition easy-to-peel seals, is used not only for the intravenous drip but also for the preparation of the injection solution if it is miniaturized. Namely, different liquid medicine can be kept in each separated compartment and the pressure can break the partition seals, just prior to its use. Thus, the injection solution can be prepared by mixing some kinds of medicine. Moreover, it can also be used in this way; a solid medicine, especially when it needs to be kept dry until it is used, such as an antibiotic, or an anticancer medicine, is filled in one separated compartment; and the other contains its solvent. At the time it is used, one breaks the intermediate seal by pressing the solvent compartment so that the solvent can flow into the medicine compartment for dissolving.

However, in these cases, the same thing happens when there is a single compartment in a soft bag, which is filled with a liquid medicine. One pierces a metallic needle to which tubing is connected, as described above, into the rubber stopper fitted to the port, which is welded on the first compartment. Thus, piercing the metallic needle on the other edge of a tube into the patient's vein may perform the injection. Generally, this kind of an injection kit has been already put to practical use. (For example, PD H2-4671, PD H6-14975).

When compared with the case of a solid medicine in a vial and its process of an injection, such as by injecting the solvent into an injection vial to dissolve medicine, and then sucking the prepared injection solution for the injection, a kit mentioned above is extremely simple. So simple that the preparation of the injection solution can be done even at a bedside. This removes the danger of making mistakes on the combination of a solid medicine and its solvent, and its quantity. Even this removes the fear of contamination, it does not mean that there is no fear of contamination at all. And also, this is restricted to an intravenous drip infusion therapy, because it is difficult to make a small quantity of the injection solution such as 1 ml, 2 ml, 5 ml, 10 ml, and 20 ml.

A simple injection device, which combines an injection needle and a port connected to a plastic solution reservoir, called “UNINJECT” (the commercial name of the product of Becton Dickinson and Company), has been known. Although, it enables to inject the liquid medicine upon its use, by pressing the reservoir, the liquid quantity of the present “UNINJECT” as described above, is so small that it is only limited to 0.1 ml˜1 ml. So, it is suitable for the special use such as vaccination of hepatitis Type B and of tetanus. Yet, it is difficult to be used under a general quantity of liquid, namely about 2˜20 ml. Moreover, it can not provide several compartments, nor prepare the injection solution by dissolving a solid medicine in the container itself, Thus, it is useful to inject a small amount of a medicine solution, which is stable as a solution.

A number of mechanism which prepare an injection solution by dissolving a solid medicine with its solvent at the time of its usage, and until when a solid medicine and its solvent is separated in a glass or a hard plastic syringe, have been reported. (For example, PD S62-14863, PD H2-58446, UD H5-152).

To be more concise, in every case, various shapes of gasket separate injection syringes, and a solid medicine is filled in one compartment with its solvent in another. At the time of its usage, one pushes forward a piston of the syringe, and the solvent is transferred through the by-pass for dissolving the medicine, by which the medicine solution is prepared in syringe and can be injected as it is. But, since every mechanism is so complicated and has difficulty to keep a medicine stable, which is unstable to humidity, so the invention has not been practically used, yet.

In the hitherto known art, there have been problems in preparation and injection of solution. Many items such as a glass vial containing a solid medicine, a glass ampoule containing solvent, an injection syringe and an injection needle should be collected. If all the devices were not ready, the injection solution can not be prepared, and so, the injection can not be performed.

Furthermore, the hitherto known art, multiple steps to prepare an injection solution; syringe and other items for injection and solution itself permit chances to spoil during its procedure. Contamination occurs easily, even in the hospital there is a danger of in-house infection, and it is almost impossible to shut out the infection outdoors.

The hitherto known art has also had a problem that during the preparation of an injection solution, the accident, such as an operator suffers a cut wound by broken glass pieces or a stub wound by a metallic injection needle, often occurs.

Further, the hitherto known art has had a problem that medical accidents caused by the mistake of the kind and/or the quantity of the medicine and/or the solvent, frequently occur. This dues to the possibilities that one can not recognize the name of the injection solution in a syringe, under the process of sucking the solution into syringe after preparation of injection solution by dissolving a solid medicine in vial.

By the hitherto known art, because used disposable injection syringes and disposable injection needle may be re-used, there have been reported many infections of AIDS or hepatitis etc. due to the re-use of used contaminated implements.

The hitherto known art has had a problem that, to discard used disposable injection syringes, disposable injection needles, glass vials and glass ampoules as medical wastes, it is necessary to classify them to many kinds, namely, glass, plastic, rubber, and metallic needles.

The hitherto known art has not been able to meet the contradictory requirements to maintain a solid medicine in dry state until use, and at the same time, to dissolve the medicine easily and speedily by the solvent in the same container, and to be ready to inject by pre-set metallic injection needle.

In comparison with a glass or a hard plastic container as a medicine container, a plastic flexible container (soft bag), has some merits such as having no damage of breaking, and being light in weight, and being easily disposed after its use. In spite of these merits, it is not used as an injection apparatus, because of the luck of mechanism of pressing the injection solution at a steady speed as can be done with a glass or a hard plastic injection syringe. By the hitherto known fact, soft bags are available as a bag for the intravenous drip infusion under a large amount of injection solution. However, the one under a small amount of the solution, such as 1˜20 ml of volume, an injection had to be practiced with a glass or a hard plastic injection syringe, transferring the solution from another container.

### OUTLINE OF THE INVENTION

[Subject to be Settled by the Invention]

With the background of a above-mentioned prior art, this invention has an object to provide an advantageous injection kit and an injection device. With this invention, the preparation of an injection solution can be made, and the injection is possible with the pre-set metallic needle, minimizing the danger of infection even in unsanitary environment, safely, easily, and speedily. By minimizing of the number of necessary materials for the preparation and injection of solution, the custody of medicaments at the pharmacy is made easy, and the kinds of used medical wastes can be limited to two kinds, namely, plastics and metallic needles.

[Means to Settle the Subject]

This invention concerns to an injection kit and an injection device that have been invented to settle the above-mentioned subjects in the result of an eager study.

That is, firstly, the injection kit of this invention is characterized by that a cylindrical multi-layer plastic bag, having 2˜4 compartments, which are separated by a welded partition easy-to-peel seal. The first compartment (medicine compartment) furnishes a nozzle outlet welded, and contains a solid medicine. The rearmost compartment (the last separated compartment of this kit) contains a liquid to dissolve the solid medicine. That is to say, a solvent and one or two middle compartment(s) contain either different kind of a solid medicine and/or its solvent. And the metallic injection needle is welded to the above said outlet, which is covered by a plastic cap until it is used. The injection kit is also characterized by that the said soft bag has the plural holes on the outside (head) and opposite (rear) to the outlet. And those holes get into to the plural cylindrical projections placed on the rear part of a saucer of the injection device, which has been described in claim 4. Thus, this invention concerns to the injection kit, and its excellent manufacturing process.

This invention is also on the injection device, which is characterized by a box-shaped container made of a transparent hard plastic to hold the soft bag of the injection kit. The container is composed of a lid and a saucer as its main body, and possesses a press block and a push out rod. The main body, a lid and a saucer, has a structure of fixing them resist to the pressure caused by pressing soft bag. A cylindrical opening in the front of the main body enables a welded nozzle-like outlet of the soft bag to stick out forward. And a round opening at the other end of the main body enables a push out rod of a press block to go through. Plural cylindrical projections exist on the back side of a saucer, which enables to fix the soft bag by getting them into plural holes on the outside and rear side edge of the soft bag. A slit placed on each side of a lid is suitable shape and size. It enables to guide the cylindrical projection on the both sides of the press block, and to press on the soft bag containing the solution, which is set in the main body with push out rod, and to move forward smoothly. A lid and a saucer have a structure of opening either vertically, on its short axis, or horizontally, on its long axis.

### BRIEF DESCRIPTION OF DRAWINGS

FIG. 1 shows a conceptual view of an example of an injection kit of this invention. (a) Shows a front view, and (b) shows a side view.

FIG. 2 shows a conceptual view of an injection kit of this invention, which is sealed to cover, by a moisture-proofing bag with gas barrier nature. (a) Shows a front view, and (b) shows a side view.

FIG. 3 shows a conceptual view of an example (vertical opening) of an injection device of this invention. (a) Shows a front view, and (b) shows a side view.

FIG. 4 shows a conceptual view of an example (horizontal opening) of an injection device of this invention. (a) Shows a front view, and (b) shows a side view.

FIG. 5 shows a conceptual view of setting an injection kit into an injection device (vertical opening) of this invention. (a) Shows a front view, and (b) shows a side view.

FIG. 6 shows a conceptual view of setting an injection kit into an injection device (horizontal opening) of this invention. (a) Shows a front view, and (b) shows a side view.

Explanation of Marks

A. solid medicine

B. solvent

a. medicine compartment

b. solvent compartment

1. partition easy-to-peel seal

2. nozzle

3. metallic injection needle

4. protective cap for an injection needle

5. multiple-layer

6. moisture-proofing, oxidation protecting bag, consisting of a gas-barrier layer

7. lid

8. saucer

9. rim of a lid 7

10. hole on rim 9 of a lid 7 (fixes with a small projection 12 on a rim 11 of a saucer 8)

11. rim of a saucer 8

12. small projection on a rim 11 of a saucer 8 (fixes with a small hole 10)

13. slit on a front side of a lid and a saucer (opening for nozzle 2)

14. projection on the inner low edge on front face of a lid (for meshing)

15. projection on the outer low edge on front face of a saucer (for meshing)

16. press block of an injection device

17. press rod of an injection device

18. hole for a press rod 17 to pass through

19. slit

20. projection on a press block 16 (fixes into a slit 19)

21. projection on the inner rear part of a saucer (fixes into a small hole of a soft bag 22)

22. small hole of a soft bag

23. layer with gas-barrier nature

24. low-melting plastic layer with gas permeability

25. Desiccant

### DETAILED DESCRIPTION OF THE INVENTION

Embodiments of this invention will be concisely explained below.

Firstly, the injection kit of this invention is explained.

The injection kit of this invention is characterized by that a welded partition easy-to-peel seal separates the cylindrically formed multi-layer flexible plastic bag (soft bag). The first compartment (a medicine compartment) contains a solid medicine, has a nozzle for an outlet of the injection solution to which a metallic needle with a protective cap is welded. The rearmost compartment, which is the furthest one from the first compartment, contains the solvent to dissolve the solid medicine.

When two kinds of solid medicine are needed, for the appropriate medical effect, and if it is preferable for them to be kept separately until the usage, two separated solid medicine compartments can be made. This can be effective especially when, for example, one of the solid medicines is easy to be oxidized and the other has a nature of oxidation. And the plural solvent compartments can be made separately, if some kinds of solvent are needed in combination, and when it is necessary to be kept apart until their usage. This is the case with a sugar solution and an amino acid, for example. Besides making the first compartment for a solid medicine, and the rearmost one for its solvent, when some other compartments are necessary between them, the compartment for a solid medicine is preferred to be made on the first compartment side, and a solvent one on the rearmost side for the arrangement order of the compartments.

The injection needle is welded to fit a nozzle outlet of the first compartment (medicine compartment) and a cap seals the injection needle to cover.

In order to avoid deterioration of a solid medicine by absorption of moisture, the medicine compartment together with a capped metallic needle can be covered with a sheath-shaped bag for the protection from moisture. Its material and the usage of desiccant are as previously explained.

The sheath-shaped bag is peeled off and removed before the injection kit of this invention is set to the injection device of this invention.

The length, the width and thickness of a soft bag differ according to the amount of the contained solid medicine and its solvent. For example, if a solid medicine is 1˜2 g and its solvent is 1 ml, 2 ml, 5 ml, 10 ml, or 20 ml, it is about 1.0˜2.5 cm wide, about 8˜15 cm long, and the thickness when a solvent is filled is under 2 cm.

Such injection kit can be made extremely effectively under the following manufacturing process.

Namely the manufacturing method is, after making some compartments separated by a welded partition easy-to-peel seal, cut off one end of a cylindrical flexible multi-layer plastic bag which has been sealed on both sides. Then after filling a solvent of, for example, 1 ml, 2 ml, 5 ml, 10 ml, or 20 ml, from the cut end, seal again by welding. After sterilization of solvent-filled bag, in an autoclave (for example, for 30˜60 minutes under the temperature of 105˜130° C.), transfer it into a clean room, and cut the other side of a medicine compartment. Fill a powdered solid medicine, produced by freeze drying method, or granule type, or a tablet, for example at the amount of 0.5, 1.0, 2.0, or 3.0 g, through the opening. A nozzle to which a capped injection needle is welded, which works as an outlet of the injection solution, is welded to cover the opening.

Another way of manufacturing is shown as follows; first, make the width of a soft bag with a welded partition easy-to-peel seal, about 6˜20 mm wider than a completed products. Then, a nozzle working as an outlet of an injection solution with a capped injection needle is welded on one side, and the other side is also welded excluding the hole, which is necessary for setting a soft bag into an injection device. Then, cut the side edge of the solvent compartment of this empty wide bag to make an opening of about 1˜2 mm long. Make the soft bag stand upward to fill the solvent through the opening. When a solvent is filled, the welding seals the opening and sealed edge is cut to leave an appropriate width of edge.

After sterilization in an autoclave, transfer it into a sterilized room, then cut the lateral end of a medicine compartment so that an opening of about 1˜2 mm wide 1˜3 cm long can be made. After a solid medicine is filled through the opening, the welding seals the opening. Then, take it out of the sterilized room and sealed edge is to leave an appropriate width of edge.

As for the injection kit manufactured under the process already shown, a sheath-shaped bag covers the medicine compartment and an injection needle to protect from moisture. Then, the bottom part of it is welded or attached at the part of a soft bag with a welded easy-to-peel seal applied.

And without making a sheath-shaped bag in advance, a desiccant may be applied or attached between the multi-layer of a transparent plastic layer with a gas barrier nature, and of a low melting point plastic layer. A powdered desiccant or a piece of paper containing desiccant may also is put between multi-layers. The multi-layer between into which a piece of paper containing desiccant is put, may hold both a medicine compartment and an injection needle of the injection kit, and its surroundings may be either welded or attached. Or the above described multi-layer may hold plural injection kits simultaneously, and the surroundings of the medicine compartment and the injection needles of every injection kit may be either welded or attached together. Putting a perforation on each kit makes a product of linked plural injection kits with each moisture-proofing bag. And such products are very useful in handle.

An injection kit with a moisture-proofing wrapping can be over-wrapped by a transparent plastic layer with a gas barrier nature.

Next, the injection device of this invention is explained.

The injection device is equipment to which an injection kit of this invention is set and the injection solution is prepared on setting, and the injection can be performed as it is. The injection device of this invention, composed of a lid part and a saucer part as its main body, and a pressing rod, can load a plastic flexible container (soft bag). The main body composed of a lid and a saucer has a mechanism of fixing the both together not to come off under the pressure inside, caused by inflation of injection kit set by pressing and pushing with pressing block by rod. A lid and saucer furnishes half round or a half cylindrical opening in front, from which a nozzle outlet of the injection solution can be projected when the kit is set. At the rear end of the device, there is the hole or the cylindrical opening, which can pass a rod that, attached to a press block. There are projections at the rear part of a saucer to fix a soft bag by getting into holes at the rear edge of the soft bag. And a press block has a rod united to it on the rear part. A press block has an appropriate shape and size to press soft bag set in the main body and to move forward smoothly.

The materials of the main body composed of a lid and saucer and a press block with a rod, may only be hygienic, light in weight, and tough. Although there may be no special restriction for the materials, metals such as stainless steel, aluminum and duralumin and a hard plastic, such as polystyrene, polyvinyl, polypropylene, ABS resins, methyl metacrylate resins, nylon resins, polyacetal, polycarbonate, etc. are suitable. A transparent hard plastic is the most suitable for the main body especially for its lid. A press block and a rod can be colored blue, red, brown and so on, so that they are easily recognized.

The size and the shape of the main body is decided by the size and shape of the soft bag loaded (repletion volume). Its size is usually about 5˜50 ml, and its shape on a transverse section is preferred to be either a rectangle or a trapezoid, or a box-shaped, with longer width than its height.

The relationship of the lid and the saucer of the main body may be like that of a lid covering a saucer, and that the both can confront with each other by making the both edge the same shape and size. The rim can be made either entirely or partly on a lid or a saucer in order to keep their confrontation. It is needless to say that it is hopeful that there is no irregularity on the surface of the inner side of a main body so as the press block to move smoothly.

As above mentioned, the mechanism of fixing the lid and the saucer, is necessary, so as not to get apart and opened under the pressure from inside which is caused by a press block slides on the soft bag set. The clasping may be a proper way of the mechanism, yet the biting of a projection and a slit may also gain its ends. The projection and its biting slit are made on a rim at the edge or on its outside of both a lid and a saucer.

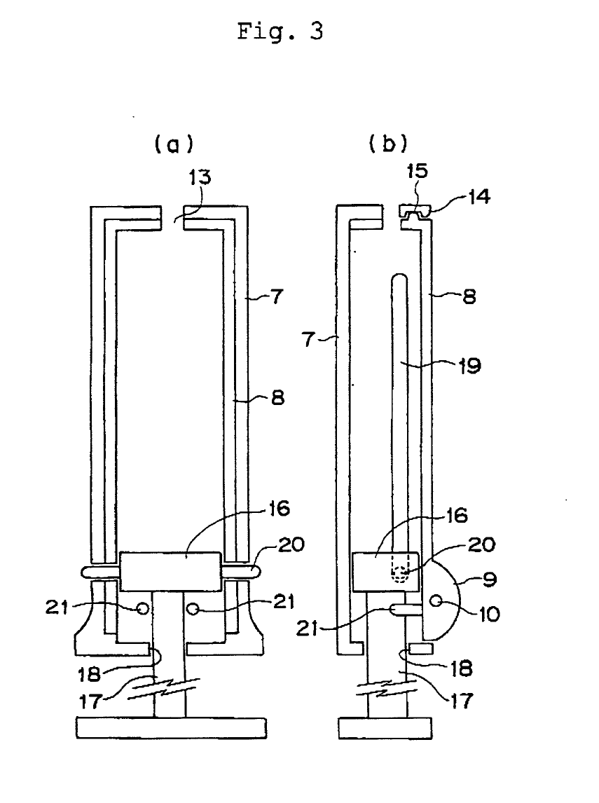

In the case of the structure that main body of injection device opens up on its short axis (vertical opening), a projection is made on the lower front part of a lid. And a projection made on the upper front part of saucer can be fixed not to open apart by biting the projection. (FIG. 3)

In this case, by making a hole on the rim at the both sides in rear part of lid, and by making a cylindrical projection on the rim at the both sides in rear part of saucer corresponding to the hole in the rim of lid, to bite each other. This can make a fulcrum of the vertical rotation.

It is also possible to make a structure of opening an injection device on its long axis (called horizontal opening). In this case, with a hinge on one side, and a clasp on the opposite side bite each other, a lid and a saucer can be fixed.

In order to operate properly, small cylindrical projections are provided on both sides of a press block, and slits are made on both sides of a lid to bite each other. A press block, which bites a slit, is made to slide back and forth by a press rod; a slit is working as a guardrail.

The height of a slit, made on the both sides of a lid, from the bottom of a saucer, is equal to the thickness of a soft bag after the container is pushed out. And the height from the lower edge of a press block to a small projection of a press block. The length of a slit is between the place, where the front edge of a press block stops when a soft bag is set in an injection device, and the place where it completely pushes out the injection solution. A scale can be put on the upper face or on the side of an injection device, in order to confirm the position of a press block.

Injection with the injection kit and with the injection device of this invention can be preformed as follows.

Namely, after the removal of a moisture-proofing sheath-shaped bag, from the soft bag, the bag is set by filling a small hole on the rear outer edge of the soft bag to a small projection, which is on the rear side of a saucer of an injection device. By pushing forward a press block a little, by pressing the compartment of the solution, a welded partition of easy-to-peel seal breaks. After it is broken, the solvent can flow into the medicine compartment to dissolve a solid medicine, and thus an injection solution is prepared.

After confirming the solid medicine is completely dissolved, the protective cap of an injection needle is removed. Then, all the air remaining in a soft bag is pushed out from the needlepoint by sliding a press block forward. Now, injection is ready.

To the patient's is skin disinfected with cotton alcohol, needle is stung, and intravenous, intra-muscular, hypodermic, or intra-dermis injection can be done as a common practice. After the injection, replace the press rod backward, open the lid, and remove used soft bag. The used soft bag is preferable to cut and separate the plastic part and the metallic needle to waste.

The injection device invented can be used repeatedly, and it is not necessary to be sterilized each time, except in special circumstances.

The above-mentioned statements are mainly the explanation on the soft bag of two compartments, which should be set to an injection device of this invention. Yet, it would be easily understood for the concerned parties that this explanation could be applied easily to the soft bag of three or four separated compartments. This device is available as an injection kit, which is similar to that of this invention, except it has one solution compartment. In this case, by pushing the rod forward, the injection solution is pushed out through the injection needle, and the injection can be done.

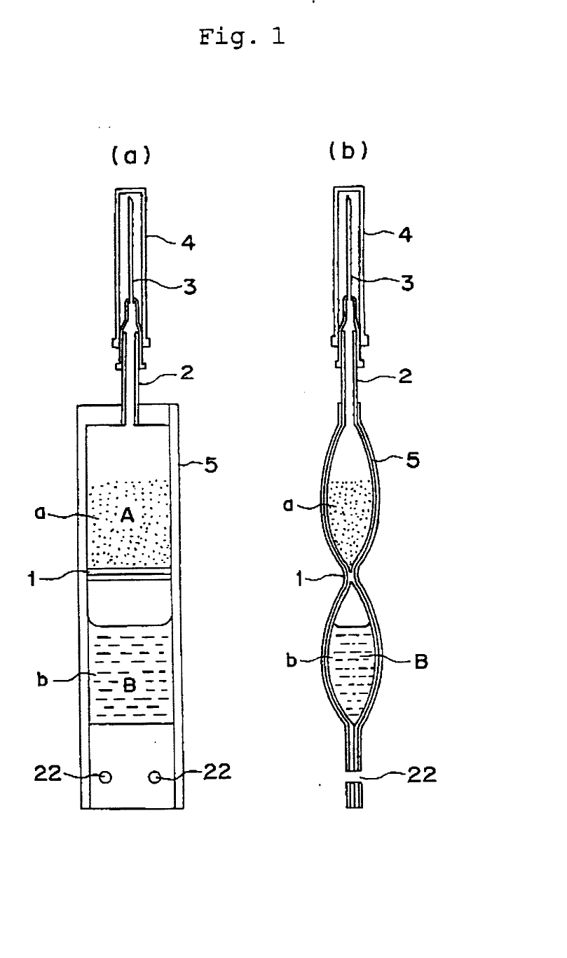

Embodiments of this invention will be explained below based on the attached drawings. FIG. 1 shows a conceptual section view of the injection kit of this invention. (a) Shows a front view, and (b) shows a side view.

The bag shown in FIG. 1 is a multi-layer cylindrical flexible plastic container (soft bag), it has the first compartment (medicine compartment) a and the second compartment (solvent compartment) b separated by a welded partition easy-to-peel seal 1. A nozzle 2 is welded to the first compartment, a metallic injection needle 3 is welded to the nozzle 2 and a protective cap 4 covers the injection needle 3.

To form a soft bag, any method including sheet-plastic method, blow-plastic method can be used.

An outlet of the injection solution which is welded to the first compartment a, can be replaced by welded a product of nozzle 2 furnishing an injection needle 3, covered by a cap 4, in advance.

The multi-layer film 5 to form the bag is produced by appropriate two or three materials, PE such as HDPE, LDPE, LLDPE, or PP or PVA and the like. It is necessary to combine two or three layers, the melting point of the most inner layer should be lower than that of the outer layer. As already known to the public, in such case, at an appropriate temperature, only the most inner layer is fused and welded to each other, thus forming an easy-to-peel seal. The plastic film used in this invention must have the resistance to heating, for example, 105˜130° C. for the sterilization of the solvent.

The medicine A to be contained in the first compartment a is a substance which is stable and tend not to change chemically at dry state, such as antibiotics, anticancer drugs, bio-active factors, peptides, proteins, various kinds of liposome. These substances may be mixed with hydrophilic substances such as low molecular weight dextrin, PEG, HES, and albumin to increase the volume of them or to stabilize them. These substances may be powder, granule, tablet, etc. prepared by the freeze-drying, reduced pressure drying, spray drying and so on to make the drugs, fine powders, to dissolve them easily. The amount of medicine has no special limitations, but 0.5˜3 g are common.

The liquid (solvent) B to be contained in the second compartment b can be, either distilled water or an aqueous solution of single and multiple mixture of salts, carbohydrates, vitamins, amino acids, proteins, etc. or fat emulsion. The quantity of them to be contained in the compartment b is, 5˜20 ml for example.

When the solid medicine should be dissolved into such a solvent combination, which is unstable, by mixing, as amino acids and carbohydrates, fat emulsion and amino acids, third compartment formed by making the second easy-to-peel partition can be used for the second solvent.

Holes 22, at the rear side of the outer edge of a soft bag, are used to make a soft bag stable by fitting to the small projections 21 on the inner rear side of the saucer 8 of an injection device.

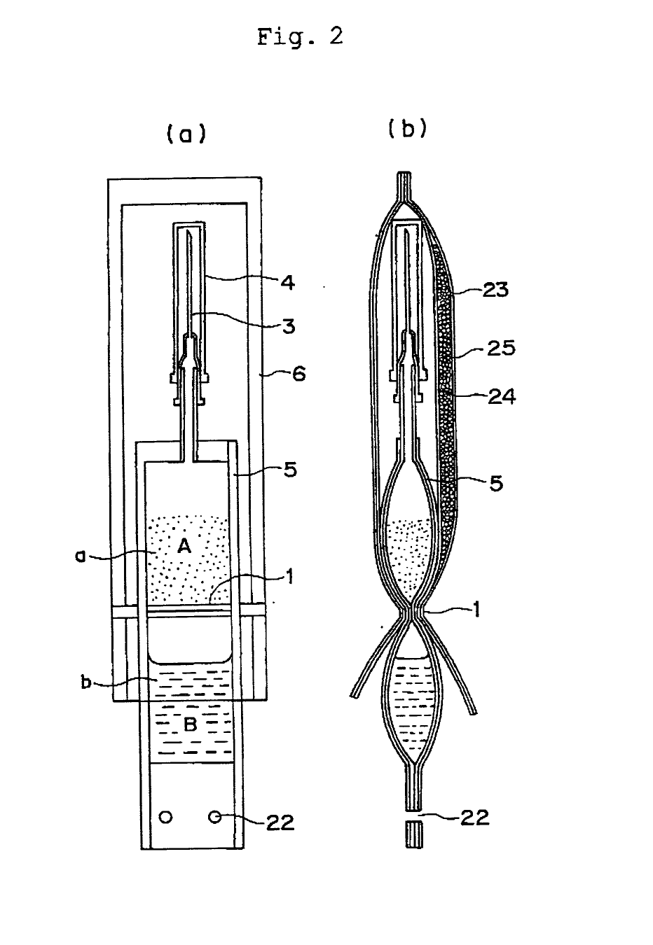

FIG. 2 shows a conceptual view of the injection KIT, in which the first compartment (medicine compartment) a furnishing an injection needle 3 covered with cap 4. These part is sealed by a moisture-proofing sheath-shaped bag 6, and one end of moisture-proof bag is either welded or attached at the part of partition of easy-to-peel seal 1 of a soft bag. (a) Shows a front view and (b) shows a side view.

A sheath-shaped bag 6 is formed by a double layer of transparent plastic sheet with a gas barrier nature 23 which is over-layered by a soft low-melting point plastic sheet with gas permeable nature 24. It has the purpose of keeping a solid medicine A in a dry condition. A desiccant 25 may either be applied or attached in the inner part of the bag. Or, desiccant is printed inside of the bag. Or a bag may be formed with the sheet of two layers 23, 24, where the desiccant 25 is included between two plastic layer. But the inner condition should be able to be well observed. Or a desiccant in an appropriate style such as desiccant in a bag may be placed in the space between the bag and the injection kit. FIG. 3 shows a conceptual view of an example of an injection device of this invention. (a) Shows its front view, and (b) shows its side view.

In this example, the main body of injection device is formed by polystyrene, a kind of a hard plastic. A lid 7 and a saucer 8 are separately formed, and the lid 7 is large enough to cover a saucer 8.

A hole 10 is formed on a rim 9, which is on the bottom of both rear sides of a lid. A rim 11, the same size with rim 9 of a lid, and is on the bottom of both rear sides of a saucer 8. (Not shown in a FIG. 3) A small cylindrical projection 12 (not described on a figure) is in the place corresponding to a hole 10 in the rim of a lid. By fixing a small projection 12 into a hole 10 of a lid, the lid opens vertically with a small projection 12 of a lid as its axis.

A vertical slit 13 is on the front face of a lid 7. It has a width, which corresponds to the external diameter of a nozzle 2 of a soft bag, and its upper part is shaped half-cylindrical. A half-cylindrical slit 13 is also on the side of a saucer 8, and it puts a nozzle 2 of a soft bag between from up and down so that an injection needle can be hold.

A projection 14 at the bottom edge of the front lid goes into a projection of a saucer 15, so that a lid and a saucer can close to be fixed.

A circular hole 18 is on the rear side of a lid 7, so that a press rod 17 of a press block 16 can pass through.

A rear side of a saucer 8 is not necessarily needed.

The traverse section shapes of a main body when a lid 7 covers a saucer 8 may be a rectangle, a circle, an oval, and so on. The depth of a lid 7 is, for example, 0.5˜2.5 cm, and the depth of a saucer 8 is, for example, 0.5˜1.5 cm.

A slit 19 is on both sides of a lid 7, and it goes along the long axis of an injection device. A small projection 20 on an axis on both sides of a press block 16 goes into it, working as a guide to slide press block 16 forward.

The distance of a slit 19 from the bottom of a saucer may equal to the place of a small projection 20 when a press block 16 presses the soft bag.

The length of a slit 19 is between the places of the rear edge of a soft bag when it is set in an injection device and the place of an outlet of a soft bag when the solution is fully pressed out of it. The width of a slit corresponds to the thickness of a projection 20 of a press block 16.

Two or three cylindrical projections 21 are provided on the rear inner bottom of a saucer 8. When a soft bag of an injection kit is set to an injection device, two or three small holes 22 on the rear outer edge of a soft bag go into the projections, thus a soft bag is fixed and stabilized when a press block 16 slides forward.

The thickness of a press block 16 may be that distance between the saucer bottom and the lid ceiling of the main body of an injection device eliminated the thickness of empty soft bag. The front view of a press block 16 is preferable to that of section of lumen, and the surface of inner side of saucer needs to be flat and smooth.

A press block 16 and a press rod 17 are desirably colored such as blue, brown, yellow, or green, so that the position of the press blockhead can be clearly seen.

The scale of volume can be printed on the injection device, in order to see the progress of a press block 16. The name of a medicine, its quantity, its concentration, and other necessary information should be printed on the surface of a soft bag, so as to be confirmed by operator's eye through a transparent plastic injection device.

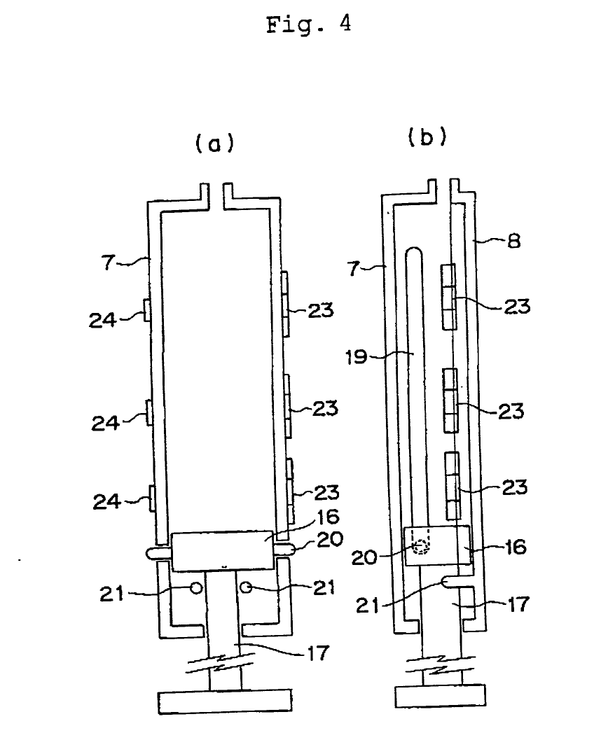

FIG. 4 shows a conceptual view of other examples of an injection device.

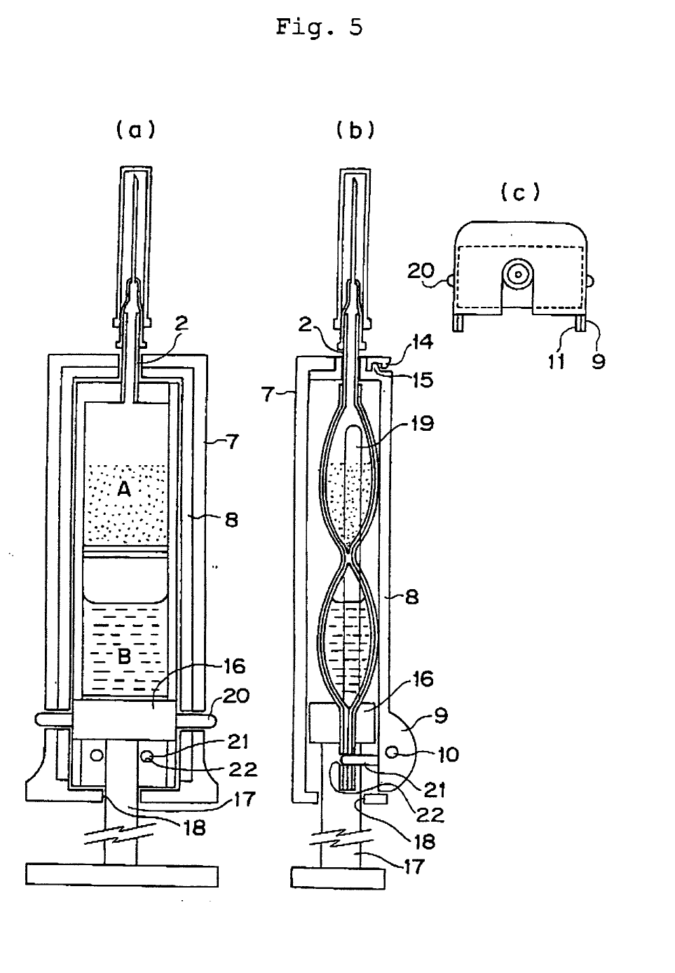

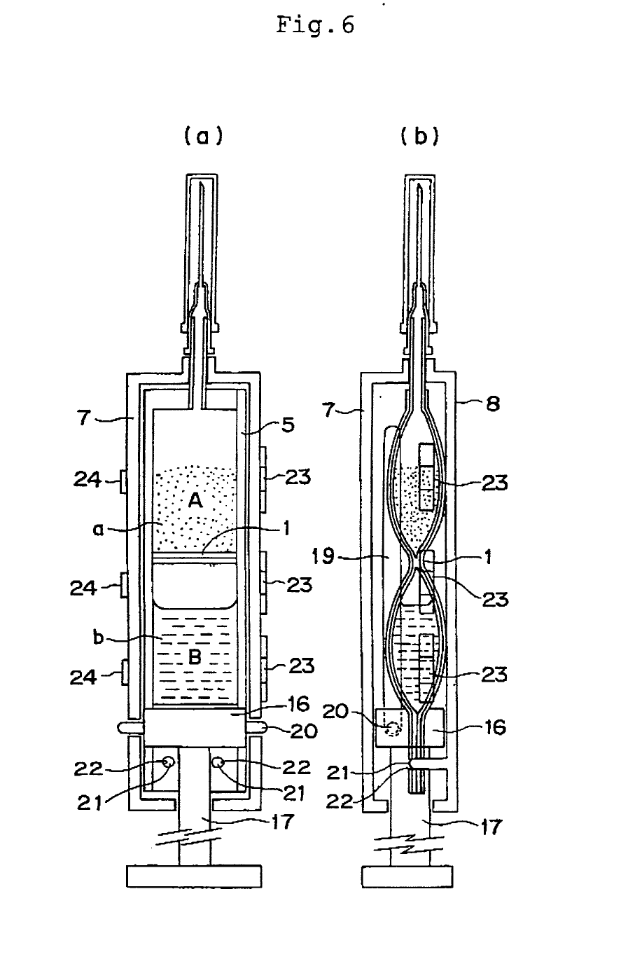

In this example, a lid 7 and a saucer 8 of an injection device face each other, and a lid 7 opens horizontally along the direction of longer axis. Thus, for example, a hinge 23 on either left or right of the connection, and a clasp 24 is provided on the opposite side of the connection. Although the projections 14 and 15 for fixing on the front lower edge of a lid and a saucer, and a rim 9 made on lower part of the rear edge of a lid and a saucer, as mentioned in the above example become unnecessary. Yet, for the other parts, it is the same as the above-mentioned example. A slit 19 is set on both of the lid 7, thus a small projection 20 on an axis of a press block 16 should be placed on upper side of press block. FIG. 5 and FIG. 6 show a conceptual views of an injection kit of this invention is set into an injection device of this invention, vertical opening and horizontal opening respectively.

[Effects of the Invention]

Special attention must be paid to the sanitary for the preparation of injection solution. By using the injection kit and the injection device of this invention, regardless of the time and place, anytime and anywhere, any operator including inexperienced person can prepare safely, easily, and speedily a sterile injection solution even in a spoiled field. And immediately after the preparation of solution, the injection can be operated by simple removal of a cap, which covers the injection needle, so that the danger of a patient suffering from infection may be greatly reduced.

Separating a multi-layer plastic flexible container (soft bag) into plural compartments by welded partition of easy-to-peel seals, and preparing an injection solution by dissolving a solid medicine with its solvent by pressing the solvent compartment upon its use, have already been put to use. Yet, with an injection kit and an injection device of this invention, an injection solution, of 1˜20 ml, can be easily prepared, and the solution can be immediately injected to a patient directly from a soft bag.

By use of this invention, any operator does not suffer from cut wound by glass pieces derived from a vial or an ampoule, and scarcely suffers from stab wound by an injection needle.

As the name of medicine, the name of solvent and the quantity or concentration of them may be printed on the surface of the soft bag, and such information on the bag can be observed easily in the injection device, even during the injection. This helps operators to recognize the name of drug and solvent, which are to handle now. This way minimizes the medical accident.

The injection kit and the injection device of this invention consist of only plastic and metallic injection needle, therefore wastes after their use are limited to only two kinds. This makes it easier to classify and dispose after use.

The injection device of this invention can be used repeatedly without sterilization except special cases, and does not need to dispose. The sterilization is not necessary every time on its used, and an operator can always carry it in his pocket.

In the injection kit of this invention, all necessary materials are furnished in one for each drug, and injection device can be used repeatedly. Therefore, the custody of drugs and injection related materials in the pharmacy simplified very much.

1. An injection kit consisting of a cylindrical plastic flexible bag (a soft bag) which is characterized by that; the bag is made by multi-layer film and is separated into 2˜4 compartments by one or plural welded partition easy-to-peel seal; the first compartment (medicine compartment) has a welded nozzle-like outlet of the injection solution and contains a solid medicine; the rearmost compartment (the last compartment of this kit) contains a solvent to dissolve the medicine; the remaining one or two mid-compartment(s) contains a solid medicine and/or its solvent; the welded nozzle-like outlet of the first compartment is welded with to a metallic injection needle, which is covered by a plastic protective cap before it is used; the above-mentioned soft bag provides the plural small holes on the outside and opposite to the outlet that get into the plural cylindrical projections placed on the rear part of a saucer of the injection device described, in order to set the soft bag to the injection device.

2. The injection kit described in claim 1 which is covered with sheath-shaped bag made from multi plastic layers consisted from a transparent gas-barrier natured plastic layer and a soft, transparent low-melting point plastic layer, is characterized by that; a desiccant is either applied or attached between the layers or the desiccant powder is enclosed or a piece of paper containing a desiccant is put between the layers; the sheath-shaped bag covering the whole compartments contains a solid medicine with the capped metallic needle. The airtightness is kept by either attaching/applying the bottom part of the sheath bag to the part of welded partition easy-to-peel seal of soft bag. Moreover, the desiccant is to be placed in the space between the sheath-shaped bag and the solid medicine compartment. The pre-described sheath-shaped bag can be removed as the soft bag is set to the injection device upon its use.

3. The manufacturing process of the injection kit pre-described in claim 1 and claim 2 is characterized by that; a material is a wide multi-layer plastic flexible bag(soft bag) of an injection kit, in which neither a solid medicine nor solvent is filled; with holes which get into the cylindrical projections on a saucer of an injection device, and with capped metallic needle welded to a nozzle of outlet of solution. Filling of the solvent into the empty solvent compartment can be done through the cut on the part of side edge of solvent compartment; after filling, then sealed by welding and sterilized. Filling of the solid medicine can be done through the cut on the part of the other side of edge of medicine compartment. After filling, sealed it by welding, and trimmed into a fixed width.

4. An injection device is characterized by that; a box-shaped container made of a transparent hard plastics, for the purpose of holding the soft bag of an injection kit described in claim 1; the container is composed of a lid and a saucer as its main body, and has a push out rod with a press block on the rearward; a lid and a saucer, has a structure of fixing the both not to be open by its own inner pressure; a hole or a cylindrical opening at the front of the main body enables a welded nozzle-like outlet of soft bag to shoot forward; a hole or a cylindrical opening at the other end of the main body enables a push out rod of a press block to go through; plural cylindrical projections exist on the back side of a saucer, which enable to fix the soft bag by getting into its plural holes made on the outside and opposite to the outlet; a slit of a suitable shape and size is placed on each side of a lid as a guide, inserting the cylindrical projection on the both sides of the press block into the slit, and pressing the push out rod enable to put pressure on the solution filled soft bag set in the main body and to move forward smoothly; a lid and a saucer have a structure of opening either vertically, on its short axis, or horizontally, on its long axis.
